(19) United States
(12) Patent Application Publication
(10) Pub. No.: US 2011/0295153 A1
(43) Pub. Date: Dec. 1, 2011

(54) LANCING DEVICE

(76) Inventors: Siew Kong Lai, Skudai Johor (MY); Chow Kian Yeo, Singapore (SG); Tze Chern Jason Lim, Singapore (SG); Peng Huay Lim, Singapore (SG)

(21) Appl. No.: 13/144,406

(22) PCT Filed: Dec. 4, 2009
(86) PCT No.: PCT/SG2009/000468

§ 371 Date: Jul. 13, 2011

(30) Foreign Application Priority Data
Dec. 4, 2008 (US) 12327817
Mar. 20, 2009 (US) 12407796

(51) Int. Cl. A61B 5/151

(52) U.S. Cl. 600/583

(57) ABSTRACT

A lancing device for use with a lancet for obtaining a blood sample is disclosed. The lancing device comprises a housing; a probe disposed in the housing; and a probe actuator for linearly displacing the probe. The probe is configured for releasably engaging a lancet and is provided with sliding surfaces for slidably engaging a pair of guides. Each sliding surface has a radius of curvature centered about a curvature defining axis, the curvature defining axis being coincident with a central longitudinal axis of the lancet when the lancet is engaged by the probe. The sliding surfaces are continually biased against the pair of guides such that when the lancet is engaged by the probe, the lancet is prevented from translating in any other direction than in a direction parallel to its central longitudinal axis during linear displacement of the probe.

### TECHNICAL FIELD

The present invention relates to a lancing device. It particularly relates to a lancing device for use with a lancet assembly and integrated with a test meter.

### BACKGROUND OF THE INVENTION

Lancing devices are typically used in the medical field to lance or break the surface of the skin on a finger, in order to extract a small blood sample for self diagnostic purposes. This may involve inserting a test strip into an analytical meter, puncturing one's finger tip with a lancing device to obtain a droplet of blood, transferring the droplet of blood onto a test element on the test strip, and checking a reading on the analytical meter for the concentration of a single analyte in the droplet of blood. The analyte may be blood glucose for a person with diabetes, cholesterol for a person with cardiovascular condition, uric acid for a person with gout, drug for monitoring effect of a therapy or presence of illegal drugs, and so on. Often, such diagnoses are repeated several times in a day and providing a diagnostic tool that is easy to operate and yet giving a less painful, if not a painfree, experience is desired.

For example, good diabetes management requires frequent monitoring of blood glucose level through self-testing. Self-testing of blood glucose is important, as it enables people with diabetes to know their blood glucose level at any time, hence allowing them to exercise tighter blood glucose control. This will help to prevent any potentially serious consequences of very high or very low blood glucose level. It is especially crucial for people who take insulin, as self-testing will allow more accurate dosage adjustment.

A lancing device is a critical tool for obtaining blood samples for glucose measurement. The primary mechanism of most lancing devices currently existing in the market, both for repeated use and disposable lancet types, involve priming a spring-based system, followed by a release of a trigger to launch the lancet or needle into the finger of the user. In this way, the lancet or needle is made to puncture a tiny hole on the finger of the user to obtain a blood sample for diagnostic purposes.

Such lancing devices generally convert potential energy from the primed spring into the kinetic energy of a moving lancet and its holder at the same time. This kinetic energy is then dissipated through impact of the lancet and its holder against a rigid stop. The rigid stop is also often used as a way of defining the depth of penetration of the needle into the finger. In most cases, potential energy from another spring is used to reverse the motion of the lancet, hence withdrawing it from the finger after the hole has been punctured.

It is quite typical to hear complaints from users of the lancing devices with design described above, in relation to pain during lancing process. This could be attributed to some of the following reasons. The lancing mechanism hitting at a hard stop at maximum velocity would cause excessive impact vibration, which will then be transmitted to the lancet. The excessive relative vibration and movement between needle and finger is likely the cause of the pain experienced by user.

Another cause of pain during lancing is an uncontrolled lancing motion of the lancet, which will result in an unpredictable trajectory of the needle during lancing process. This uncontrolled motion refers to the ability of the lancet and its holder to move within the sliding clearance offered by its guides, which are often plastic molded features. In addition to that, impact noise is perceived as pain most of the time, since it forms part of the overall user experience. Devices with such lancing mechanism, which relies on impact to define the lancet's penetration depth and to reverse its motion, are often perceived by the user as being noisy and painful.

Examples of lancing device with a design intended to allow less painful blood withdrawal, may be seen in the following U.S. patents. U.S. Pat. No. 4,924,879 discloses a blood lancing device, which convert the relaxation movement of the drive spring by means of a rotatable drive rotor into the prick movement, hence allowing blood withdrawal with little or no pain. The vibration caused by the impact of the lancet holder onto a hard stop can then be avoided. The rotor is driven by a coaxial coil spring and the rotation movement of the rotor is converted to the linear movement of the lancet by means of a push rod system.

U.S. Pat. No. 5,318,584 discloses a lancing device with the drive rotor having a rotation axis parallel to the prick direction and is also driven by a coaxial coil spring. The conversion of the rotational movement into the necessary linear movement of the lancet holder is performed by a rotary drive. The design allows a very good pricking behavior with low vibrations and a reproducible pricking depth, hence resulting in less pain.

U.S. Pat. No. 4,203,446 discloses a spring lancet holder with improved accuracy and reproducibility of puncture wounds in the skins by minimizing the recoil transmitted to the lancet holder by actuation of the drive mechanism, which pushes the lancet into the skin.

U.S. Pat. No. 7,396,334, assigned to Roche Diagnostics Operations, Inc., describes a needle and lancet body integral with a test element. The accompanying figures show the tip of the needle is embedded in an elastic material whilst the drive end of the needle extends from the rear of the lancet body.

US Publication No. 2008/0262386, also assigned to Roche Diagnostics Operations, Inc., describes an analytical system for detecting an analyte in a body fluid, and a disposable integrated puncturing and analyzing element. The instrument is cheap to manufacture and allows a user full control over the individual steps in collecting a blood sample for analysis.

US Publication No. 2008/058631, assigned to Beckton Dickinson, describes a blood glucose meter having integral lancet device and test strip storage vial for single hand use. By combining these multiple components into a single device, the glucose meter requires fewer steps in its use.

Despite development in the art, there still exists a need for a device and method for analyzing a person's physiologic fluid for a medical condition that overcome the shortcomings of known devices.

### SUMMARY OF THE INVENTION

According to a first aspect, there is provided lancing device for use with a lancet for obtaining a blood sample. The lancing device comprises a housing; a probe disposed in the housing; and a probe actuator for linearly displacing the probe. The probe is configured for releasably engaging a lancet. The probe is provided with sliding surfaces for slidably engaging a pair of guides; wherein each sliding surface has a radius of curvature centred about a curvature defining axis, the curvature defining axis being coincident with a central longitudinal axis of the lancet when the lancet is engaged by the probe; and wherein the sliding surfaces are continually biased against the pair of guides such that when the lancet is engaged by the probe, the lancet is prevented from translating in any other direction than in a direction parallel to its central longitudinal axis during linear displacement of the probe.

The pair of guides preferably comprises two sloped surfaces, each of the sloped surfaces being disposed on either side of the curvature defining axis.

The probe, the probe actuator and the pair of guides may be disposed on a base plate of the housing. The base plate is preferably moveable relative to the housing in a direction parallel to the central longitudinal axis of the lancet for adjusting the protrusion of the lancet from the lancet assembly during displacement of the probe when the probe is engaged with the lancet.

The lancing device preferably further comprises a second pair of guides and further sliding surfaces provided on the probe for slidably engaging the second pair of guides.

The probe actuator is preferably rotatable about a pivot disposed on the base plate of the housing with an axis of rotation perpendicular to the central longitudinal axis of the lancet.

The probe actuator preferably comprises a driving pin disposed at a distance from the axis of rotation for engaging a driving slot provided in the probe, the driving slot being configured such that rotation of the probe actuator in a driving direction results in linear displacement of the probe.

The lancing device may further comprise a priming slot provided in the probe for accommodating the driving pin therein during rotation of the probe actuator to a primed position, the priming slot being configured such that rotation of the probe actuator to the primed position results in no linear displacement of the probe.

The lancing device preferably further comprises a priming actuator for rotating the probe actuator to the primed position, a biasing element for biasing the probe actuator towards rotating in the driving direction, and an actuating button configured for releasing the probe actuator from the primed position such that when the actuating button is pressed, the probe actuator rotates in the driving direction under the bias of the biasing element.

The lancing device may further comprise means for minimizing vibration of the lancet during linear displacement of the probe when engaged with the lancet.

The housing is preferably configured for removably attaching a disposable lancet assembly thereto, the disposable lancet assembly comprising the lancet, and wherein the probe is configured for releasably engaging the lancet when the lancet assembly is attached to the housing.

The lancing device may further comprise an analyte test meter for determining concentration of an analyte in the blood sample.

According to a second aspect, there is provided a disposable lancet assembly for use with a lancing device. The lancet assembly comprises a casing configured for releasable attachment to a housing of the lancing device; a lancet contained in the casing, the lancet being configured for releasable engagement with a linearly displaceable probe of the lancing device, the lancet being moveable with respect to the casing when the lancet is engaged with the linearly displaceable probe; and a test strip disposed on the casing for receiving a blood sample thereon.

The disposable lancet assembly may comprise locking adaptations on the casing and on the lancet body for preventing egress of a tip of the lancet from the casing after use. The test strip may comprise a sensing end for receiving the blood sample and a terminal end for contacting sensing terminals of a test meter provided with the lancing device.

According to a third aspect, there is provided a lancing kit comprising the lancing device and the disposable lancet assembly mentioned above.

According to a fourth aspect, there is provided a method for determining an analyte in a blood sample. The method comprises inserting a lancet assembly into an integrated device comprising a lancing device and a test meter, the lancet assembly comprising a lancet disposed within a casing and a test strip disposed on the casing, said lancet and casing comprising locking adaptations for preventing egress of a tip of the lancet from the casing after use; actuating the lancing device to lance a finger with the lancet; collecting a blood sample from the lanced finger; transferring the blood sample onto the test strip and obtaining a reading from the test meter; and removing the lancet assembly from the integrated device and simultaneously locking the lancet within the casing.

### BRIEF DESCRIPTION OF THE DRAWINGS

Exemplary embodiments will now be described with reference to the accompanying drawings, by way of example only, in which:

FIG. 1 is a perspective view of a first exemplary embodiment of a lancing device according to the present invention;

FIG. 2 is a top perspective view of a top case of the lancing device of FIG. 1;

FIG. 3 is an exploded bottom or underside view of the top case of FIG. 2;

FIG. 4 is an exploded view of a bottom case of the lancing device of FIG. 1;

FIG. 5 is a top perspective view of a probe with cam profile of the lancing device of FIG. 1;

FIG. 6 is a bottom perspective view of the probe of FIG. 5;

FIGS. 7 a-b show lancet displacement profiles in z-x and z-y axes of the lancing device of FIG. 1 and its competitor;

FIG. 8 is a top perspective view of a probe actuator of the lancing device of FIG. 1;

FIG. 9 is a bottom perspective view of the probe actuator of FIG. 8;

FIG. 10 is a cross sectional view of a connection between the probe actuator of FIG. 8 and the bottom case of FIG. 4;

FIG. 11 is a top perspective view of a final assembly of the lancing device of FIG. 1;

FIG. 12 is a perspective view of an integrated lancing and testing device according to a second exemplary embodiment of the invention;

FIG. 13 is a perspective view of a lancet assembly formed integrally with an analyte test strip for use with integrated device of FIG. 12;

FIG. 14 is an exploded perspective view of the lancet assembly of FIG. 13;

FIG. 15 is a perspective view of a lancet of the lancet assembly shown in FIG. 14;

FIG. 16 is a sectional view of the lancet assembly of FIG. 13;

FIG. 17 is a partial sectional view of the lancet assembly coupled to a probe and test terminals of the integrated device of FIG. 12;

FIG. 18 is a perspective view of a second exemplary embodiment of a lancet;

FIG. 19 is an exploded perspective view of a second exemplary embodiment of a lancet assembly incorporating the lancet of FIG. 18;

FIG. 20 is a sectional perspective view of the lancet assembly of FIG. 19;

FIG. 21 is a top perspective view of an integrated device according to a preferred alternative embodiment of the invention;

FIG. 22 is a top perspective view of a lancing mechanism of the integrated device of FIG. 21;

FIG. 23A is an exploded assembly view of a lancet assembly according to a preferred alternative embodiment of the invention;

FIG. 23B is a perspective view of the lancet assembly of FIG. 23A,

FIGS. 24A to 24G illustrate a sequence of steps for using the lancet assembly and integrated device of FIG. 12 or FIG. 21; and

FIG. 25 shows a schematic end view of a configuration of a pair of guides and sliding surfaces on a probe of the device of FIG. 1, FIG. 12 or FIG. 22.

### DETAILED DESCRIPTION OF THE EMBODIMENTS

Description of embodiments of the present invention shall now be explained in detail, with reference to the attached drawings. It is to be understood that no limitation of the scope of the invention is thereby intended, such alterations and further modifications in the illustrated device, and such further applications of the principles of the invention as illustrated therein being contemplated as would normally occur to one skilled in the art to which the invention relates.

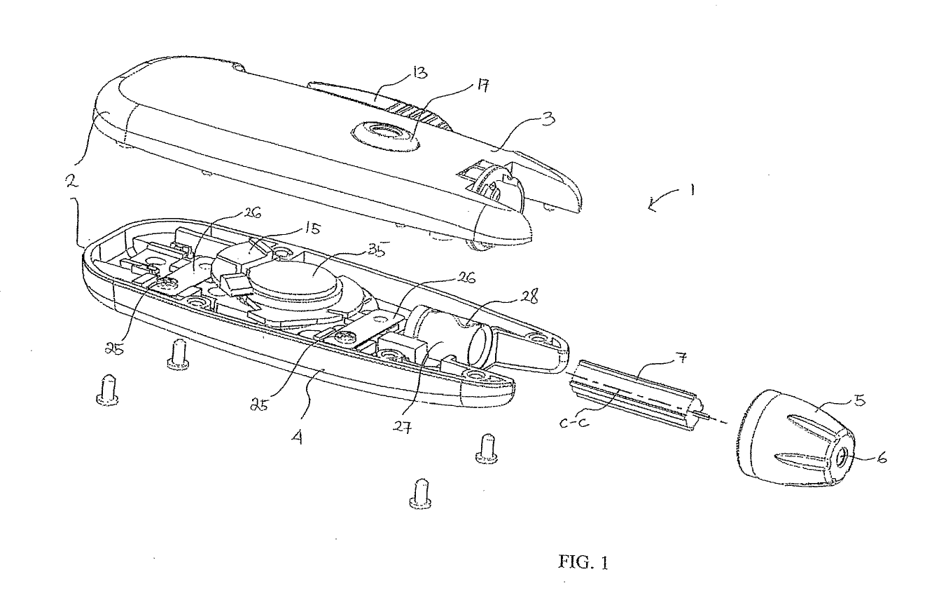

FIG. 1 shows a perspective view of a lancing device 1 according to a first embodiment of the invention. In this embodiment, a housing 2 of the lancing device 1 comprises a top case 3 and a bottom case or base plate 4. Screws are used to hold the top case 3 and bottom case or base plate 4 together. In another embodiment, the top case 3 and bottom case or base plate 4 may be joined together by ultrasonic welding. A cap 5 is preferably disposed at a front end of the housing 2 which has an opening 6 for exit and reentry of a lancet 7 having a central longitudinal axis C-C. The top case 3 and bottom case or base plate 4 are preferably configured to together form an oval shaped housing 2 for easy handling by a user.

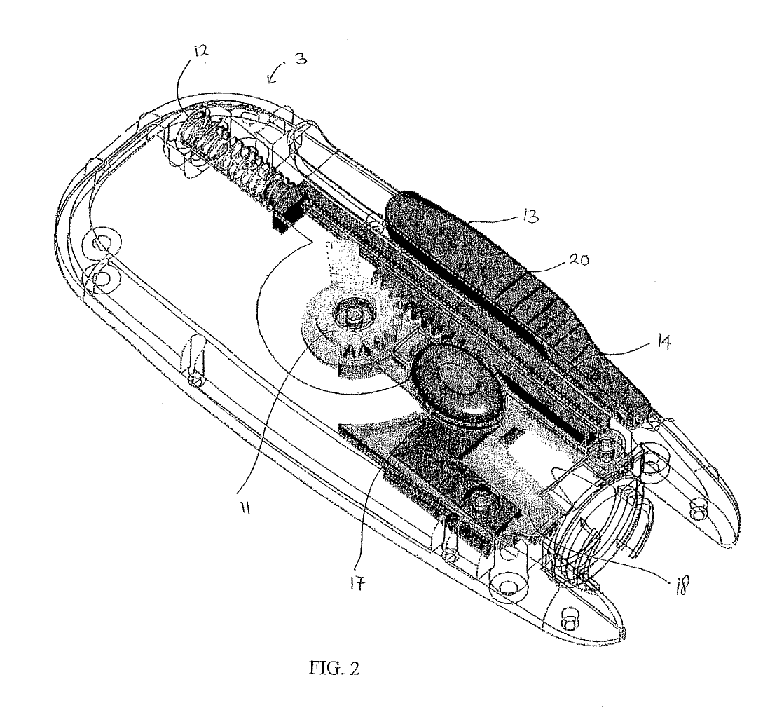

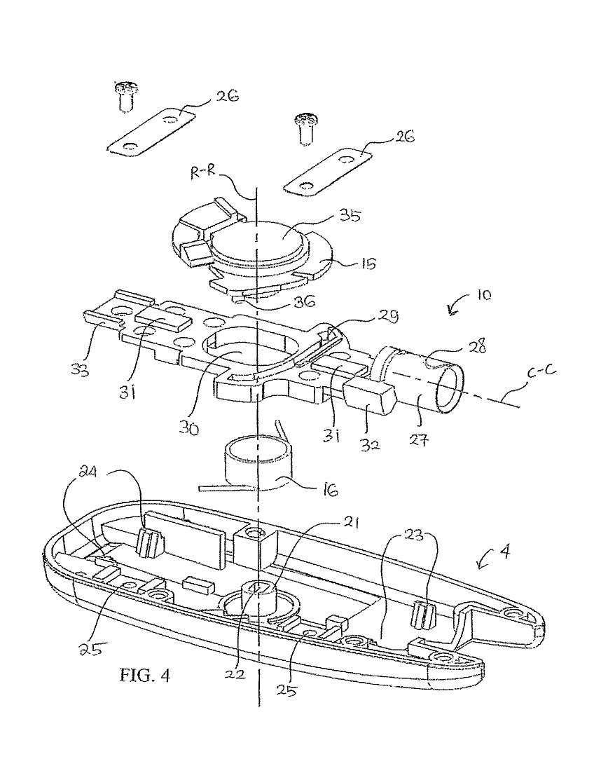

FIG. 2 shows a top perspective view of the layout of the top case 3 assembly of the lancing device 1 while details of the layout of the bottom case or base plate 4 of the lancing device 1 are illustrated in FIG. 4. The bottom case or base plate 4 comprises a guide pin or pivot 21 projecting upwardly from the middle of the bottom case or base plate 4. The guide pin or pivot 21 has a frame slot 22 for engaging a damper 35 provided with the probe actuator 15. A priming system in the lancing device 1 comprises a priming gear 11, a rack 14 engaging the priming gear 11, a compression spring 12 for biasing the rack 14 towards a rest position, a priming button or priming actuator 13, a probe actuator 15 coupled to the priming gear 11, a torsion spring 16 attached to a pivot or guide pin 21 on the bottom case 4 and to which the probe actuator 15 is rotatably attached, and a fire button or actuating button 17 for releasing the probe actuator 15 from a prime position back to a rest position.

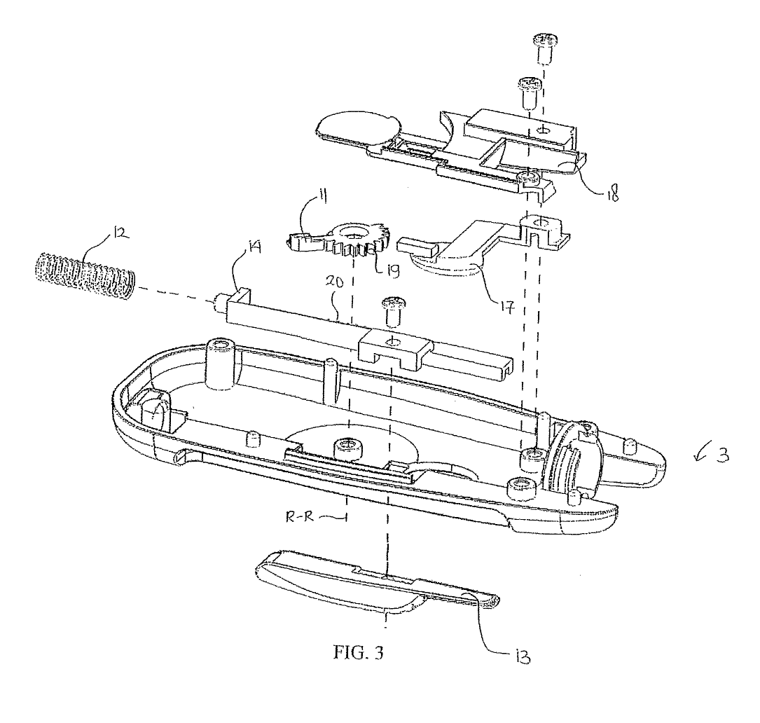

Details of the configuration of the priming system are further illustrated in FIG. 3 which shows an exploded bottom or underside view of the top case 3 of the lancing device 1. In this embodiment, a top case cover or securing plate 18 connects the priming gear 11 and the fire button 17 to the top case 3 via screws connection. The priming gear 11 is rotatable with respect to the top case 3 and is coupled to the rack 14 in such a way that the teeth 19 of the priming gear 11 engage the teeth 20 of the rack. 14.

The priming button or priming actuator 13 is connected to the rack 14 via screws connection, as illustrated in FIG. 3. When a user pulls the priming button 13 downward or towards a rear end of the housing 2, the rack 14 is brought towards the rear end of the housing 2 to a stop position, thereby causing the priming gear 11 to rotate about an axis of rotation R-R in a counter-clockwise direction when viewed from above. As the priming gear 11 rotates, it engages the probe actuator 15 such that rotation of the priming gear 11 rotates the probe actuator 15 about the axis of rotation R-R in a similar counter-clockwise direction as the priming gear 11 so as to prime the probe actuator 15 against the bias of the torsion spring 16.

Upon rotation of the probe actuator 15 to the primed position, the probe actuator 15, together with the torsion spring 16 where potential energy is stored, are locked in the primed position and will only be released once the fire button 17 is pressed. The compression spring 12 returns the priming button 13 and the priming gear 11 back to their original rest positions after the probe actuator 15 has been rotated to the primed position. Once the fire button 17 is pressed, stored potential energy in the torsion spring 16 is imparted to the probe actuator 15 to rotate the probe actuator 15 in a driving direction back to its rest position, preferably clockwise viewed from above. The torsion spring 16 thus serves as a biasing element for biasing the probe actuator 15 towards rotating in the driving direction.

Rotation of the probe actuator 15 in the driving direction linearly displaces a probe 10 with a cam profile 29 to which the probe actuator 15 is moveably engaged, resulting in forward sliding of the probe 10 together with a lancet 7 engaged by the probe 10 towards the skin of the user. As can be seen, the axis of rotation R-R of the priming gear 11 and probe actuator 15 is perpendicular to the central longitudinal axis C-C of the lancet 7 when the lancet 7 is engaged by the probe 10.

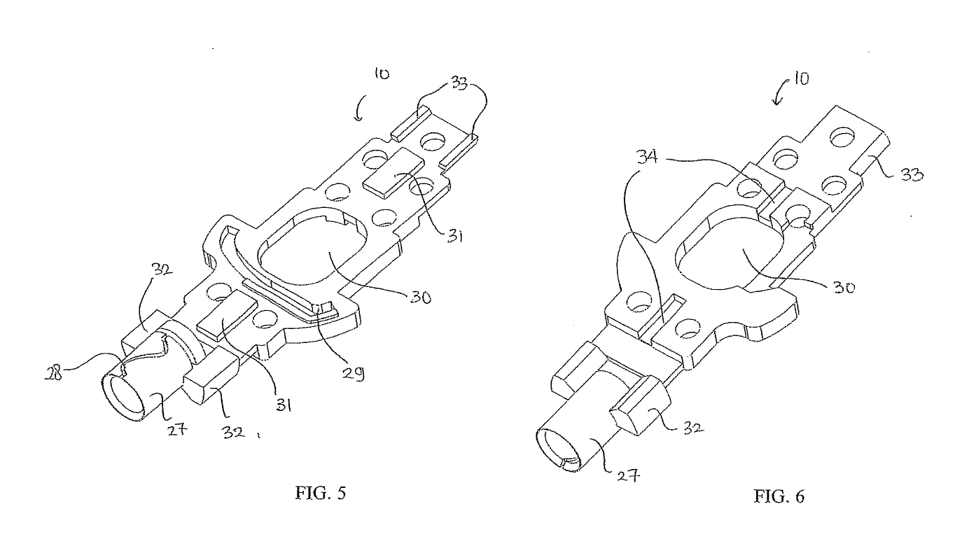

FIG. 5 illustrates a perspective top view of the probe 10 according to the first embodiment of the invention. The probe 10 may be provided with a collar 27 disposed at a front end of the probe 10 for engaging the lancet 7. The collar 27 has a cutaway 28 for providing a gripping force to the lancet 7. The cutaway 28 offers the flexibility to open up the collar 27 after the lancet 7 is inserted. Hence, the lancet 7 can be easily removed and replaced accordingly. The probe 10 has a slotted opening 30 in the middle part for receiving the probe actuator 15 therethrough. Two raised pads 31 are preferably provided on an upper surface of the probe 10 for contacting leaf springs 26. The raised pads 31 are preferably positioned before and after the slotted opening 30 respectively. FIG. 6 shows a perspective bottom view of the probe with cam profile 10. Slotted guides 34 are provided in the front and rear part of the probe 10 for limiting rotational movement of the probe 10 during lancing.

Referring to FIGS. 3 to 9, the cam profile 29 of the probe 10 is integrally formed or molded into the probe 10 to create a driving slot 29 in the probe 10. A driving pin or cam follower 36 is provided on the probe actuator 15 for engaging the driving slot 29 provided in the probe 10. The driving pin 36 is disposed at a distance from the axis of rotation of the probe actuator 15. The driving slot 29 is configured to retain the driving pin 36 within the driving slot 29 during rotation of the probe actuator 15, such that rotation of the probe actuator 15 in a driving direction results in linear displacement of the probe 10 as a consequence of the driving pin 36 accurately tracing the surface of the cam profile 29.

The cam profile or driving slot 29 is responsible for regulating the speed of the lancet 7 when the lancet 7 is engaged by the probe 10 and the fire button 17 is pressed. The velocity profile of the lancet 7 is controlled by the cam profile 29. In other words, appropriate contouring of the cam profile 29 allows the related lancet displacement and velocity profile to be optimized for minimum pain and enhanced user compliance.

Preferably, the lancet 7 penetrates the skin relatively quickly but decelerates smoothly and gradually to zero velocity at a maximum depth of penetration into the target area on the finger, where nerve endings are abundant. Smooth transition to zero velocity and absence or reduction of vibration of the lancet 7 reduces pain to the user. Slow and controlled retraction of the lancet 7 prevents the wound channel from collapsing and allows blood to flow directly to the surface of the skin. This encourages rapid healing of the puncture wound and offers a less painful lancing experience to the user at the same time.

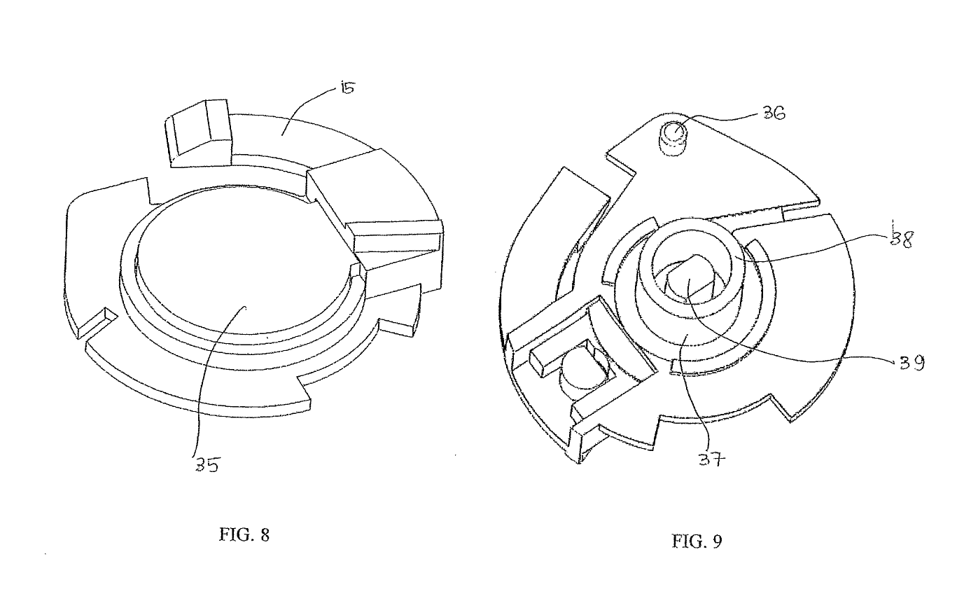

FIG. 8 and FIG. 9 illustrate a perspective top and bottom view of the probe actuator 15. The probe actuator 15 is provided with a damper 35 positioned in a middle part of the probe actuator 15. The damper 35 is provided for minimizing vibration of the lancet 7 during linear displacement of the probe 10 when engaged with the lancet 7. A frame slot 37 is provided in the middle of the damper 35 and comprises two different parts; an outer circular ring 38 for capping over the pivot or guide pin 21 on the bottom case or base plate 4, and a small centrally located protrusion 39 disposed inside the circular ring 38 for inserting the damper 35 into the frame slot 22 of the pivot or guide pin 21 of the base plate 4.

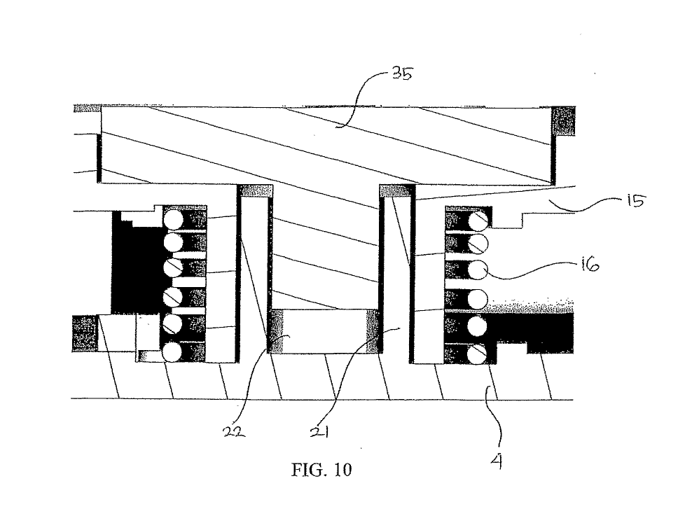

The connection between the damper 35, the probe actuator 15, the torsion spring 16 and the pivot or guide pin 21 of the bottom case or base plate 4 is further illustrated in FIG. 10. The inner surface of the guide pin 21 interacts with the damper 35, whereas the outer surface of the guide pin 21 interfaces with the circular ring 38 of the probe actuator 15, providing guidance for the probe 10. The torsion spring 16 is positioned to be resting on an outer surface of the probe actuator 15. Hence, the kinetic energy of the propelling lancet of the present invention is not dissipated through impact but rather through the damper 35. This configuration will minimize or even eliminate the noise produced during the lancing process and will enhance the user's compliance significantly.

Besides the cam profile 29, the ratio of the damper 35 and the stiffness of the torsion spring 16 are other factors that determine the velocity profile of the lancet 7. It is preferred if the torsion spring 16 is not too stiff, as it will require more effort from the user to prime it. The use of a less stiff spring is compensated by proportionally reducing the damping provided by the damper. The damping effect can be appropriately adjusted by using a damper of different size. The cam profile 29 determines how much of the potential energy from the torsion spring 16 is converted to the kinetic energy of the lancet 7. In summary, the combination of the effect of different cam profile 29, different stiffness of the torsion spring 16 and different ratio of the damper 35 can be optimized for achieving desired velocity profile of the lancet 7.

As shown in FIG. 4, the base plate or bottom case 4 is further provided with two pairs of guides 23 and 24 preferably having a v-shaped profile disposed on the front and rear part of the bottom case or base plate 4 for locating the probe 10 thereon. Each pair of guides 23 and 24 comprises two sloped surfaces facing upwardly and inwardly for slidably engaging sliding surfaces or profile slides 32 and 33 provided on the probe 10. Preferably, the front profile slides 32 are slightly larger than the rear profile slides 33.

The sliding surfaces or the profile slides 32 and 33 have circular profiles whose centre axes coincide with that of the lancet centre. In other words, each of the sliding surfaces 32, 33 has a radius of curvature IT centred about a curvature defining axis CDA as shown in FIG. 25, wherein the curvature defining axis CDA is coincident with the central longitudinal axis C-C of the lancet 7 when the lancet 7 is engaged by the probe 10. The sliding surfaces 32, 33 therefore comprise sections of a right circular cylinder RCC sharing a same central longitudinal axis C-C as the lancet 7.

Two pin connections 25 located on the bottom case or base plate 4 are provided for attachment of a leaf spring 26 each. The leaf springs 26 act on the probe 10 to bias the sliding surfaces 32 and 33 of the probe 10 against the guides 23 and 24, such that movement of the probe 10 in z-axis or in a direction perpendicular to the central longitudinal axis of the lancet 7 is eliminated or minimized during sliding or movement of the lancet 7 when engaged with the probe 10. The leaf springs 26 thus ensure that the probe 10 is always be in contact with the v-shaped profile guides 23 and 24 of the bottom case or base plate 4, thereby minimizing pitching of the lancet 7 during lancing, hence reducing pain.

As a result of the curvature defining axis CDA of the sliding surfaces 32, 33 being coincident with the central longitudinal axis C-C of the lancet 7, and also the action of the leaf springs 26 biasing the sliding surfaces 32, 33 against the guides 23, 24, the lancet 7 is prevented from translating in any other direction than in a direction parallel to its central longitudinal axis C-C during linear displacement of the probe 10. This will limit the probe movement, if any, to a slight minimum rotation of the lancet 7, instead of lateral movement of the lancet 7, thus reducing any pain experienced by the user to a minimum.

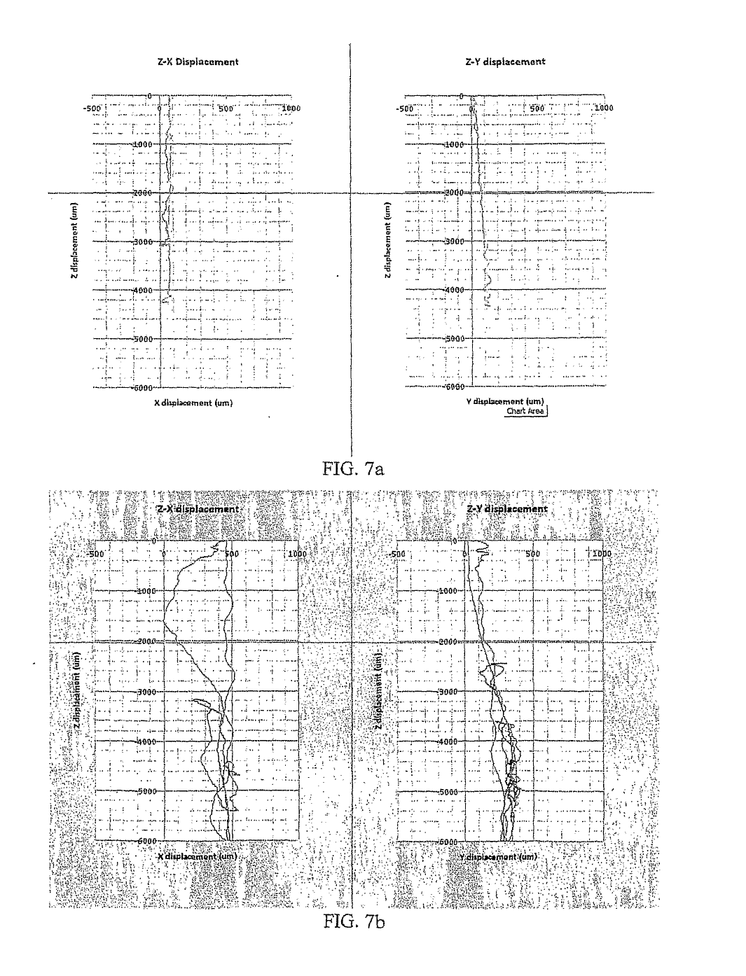

FIGS. 7 a-b show the comparison of the lancet displacement in z-x and z-y axes during the lancing process, between the lancing device of the present invention (FIG. 7a) and a leading lancet product (FIG. 7b). The displacement profiles clearly show that there is minimum or no lateral movement of the lancet 7 engaged by the lancing device 1 during lancing. The curved sliding surfaces 32 and 33 acting against the guides 23 and 24 enhance controlled motion of the lancet 7 for its entry and withdrawal from the skin of the user during lancing, as shown by an almost straight line in its displacement profiles indicating little or no lateral movement of the lancet 7 during its entire lancing trajectory. This feature allows the pain experienced by the user during lancing to be reduced to a minimum and serves as a significant improvement over the competitor's product as it ensures that the only freedom of movement allowed for the probe 10 is rotation of the probe 10 about the central longitudinal axis of the lancet 7. The clearance between the slotted guides 34 and the pivot or guide pin 21 determines the extent of rotation of the probe 10. The profile slides or sliding surfaces 32 and 33 sit on the v-shaped profile guides 23 and 24, rotating with the same center of rotation as that of the lancet 7, while the probe 10 slides forward and backward during lancing. Pitching and vibration of the lancet 7 are therefore minimized during lancing. This means that the lancet 7 is always guided, without any sliding clearance for translation in any other direction than the direction parallel to its central longitudinal axis. The only allowed freedom of motion for the lancet 7 during sliding is rotation about its central longitudinal axis.

After lancing, the lancet is retracted from the skin of the user as the probe 10 slides backward as the cam follower 36 of the probe actuator 15 continues to move within the driving slot 29 along the cam profile 29 that is embedded or integrally formed in the probe 10. A top perspective view of the final assembly of the lancing device 1 according to the first exemplary embodiment of the invention is shown in FIG. 11.

A second exemplary embodiment of a lancing device according to the present invention is shown in FIG. 12. In this embodiment, an integrated lancing and testing device 150 is provided comprising a removable lancet device or lancet assembly 100 and a test meter 170 that includes the lancing device. The test meter 170 including the lancing device together form an integrated device 170 used with the removable lancet assembly 100. The lancet device or lancet assembly 100 is an assembly of a lancet 70, a casing 40 and an analyte test strip 60. As shown in FIG. 13, the lancet 70 is disposed inside the casing 40 and the test strip 60 is disposed on the casing 40. FIG. 14 shows an exploded view of the lancet assembly 100. As shown in FIGS. 13 and 14, each test strip 60 has a terminal end 62 and an analyte sensing end 64 opposite the terminal end 62.

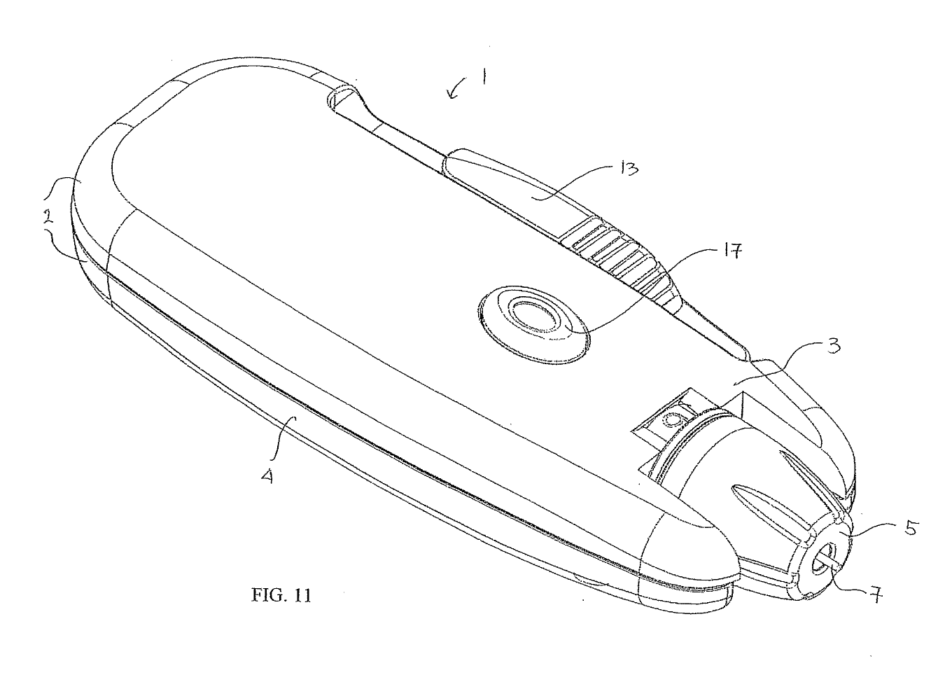

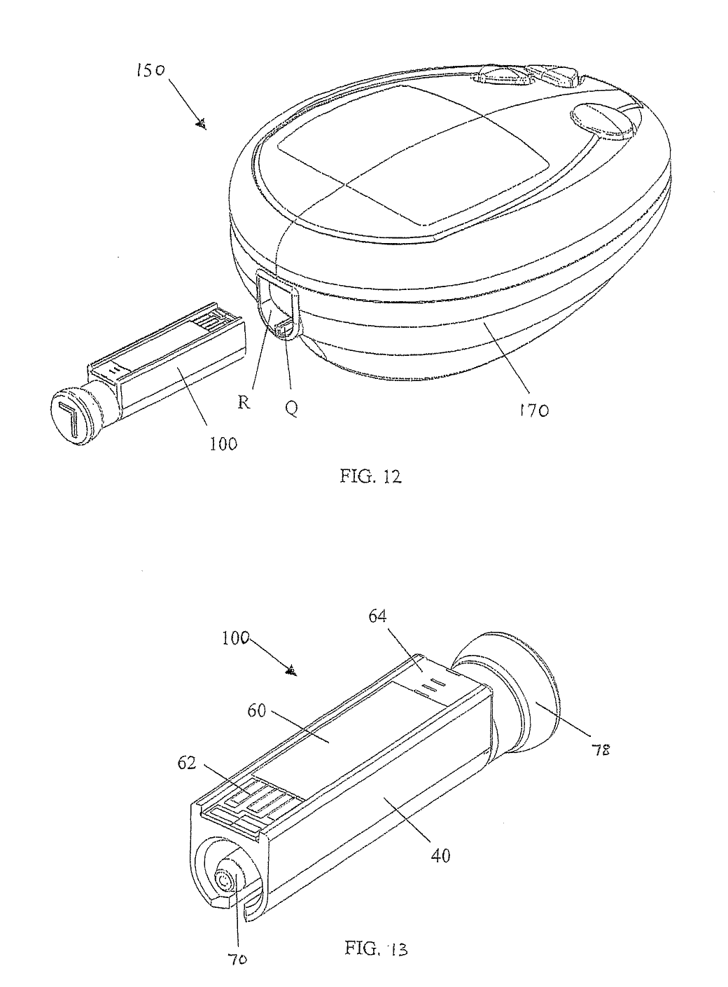

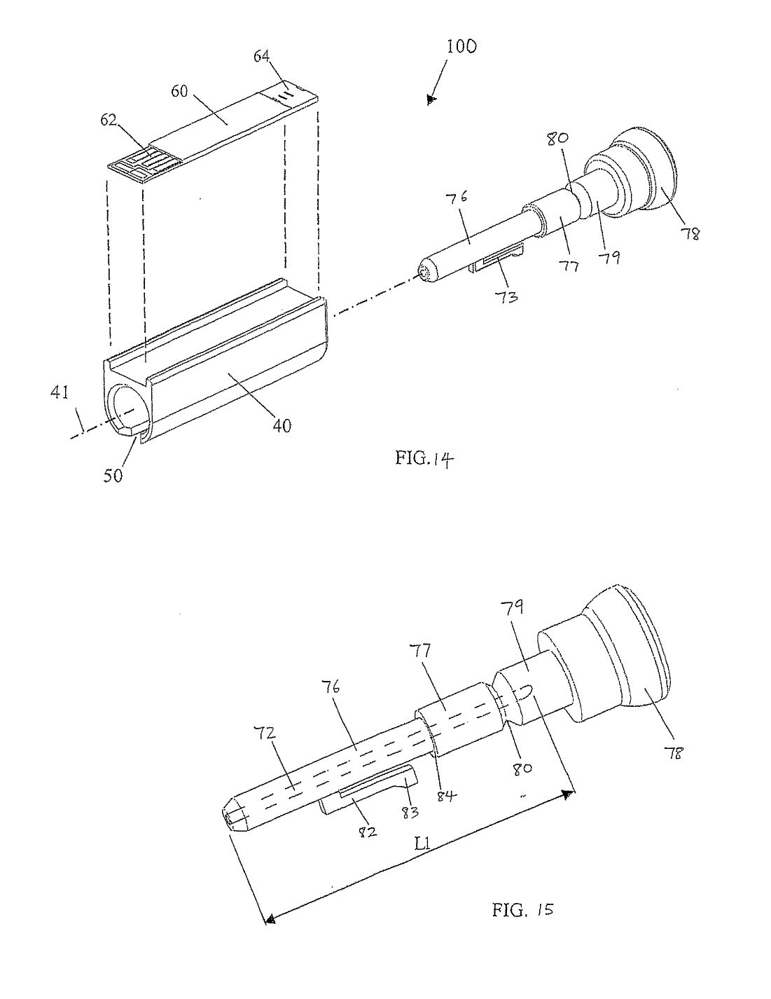

As shown in FIG. 15, the lancet 70 of the lancet assembly 100 includes a needle 72 that is sterilized and insert molded with a thermoplastic such that the needle's pointed end 74 is molded within a cap 78. As shown in FIG. 15, the cap 78 is connected to the molded body of the lancet 70 by a notch 80 of reduced cross-section. In between the cap 78 and the notch 80 is a locating part 79. The locating part 79 is concentric with the needle 72. The molded lancet body is made up of two cylindrical parts 76, 77 with an end of the larger sectional part 77 partly forming the notch 80 and the joint with the smaller sectional part 76 forming a step 84. Projecting from the cylindrical surface of the smaller sectional part 76 is an L-shaped catch 82. The L-shaped catch 82 has an arm 83 pointing in the same direction as the needle pointed end or lancet tip 24. The free end of the lancet body 76 is chamfered for easier insertion into the collar 27 of a probe 10 of the lancing device in the integrated device 170. In one embodiment, the length from the free end of the lancet body 76 to the needle's pointed end 74 is L1; a corresponding length of the needle's pointed end 74 to a front end or face 42 of the casing 40 is L2. Length L2 is more clearly seen in FIG. 17.

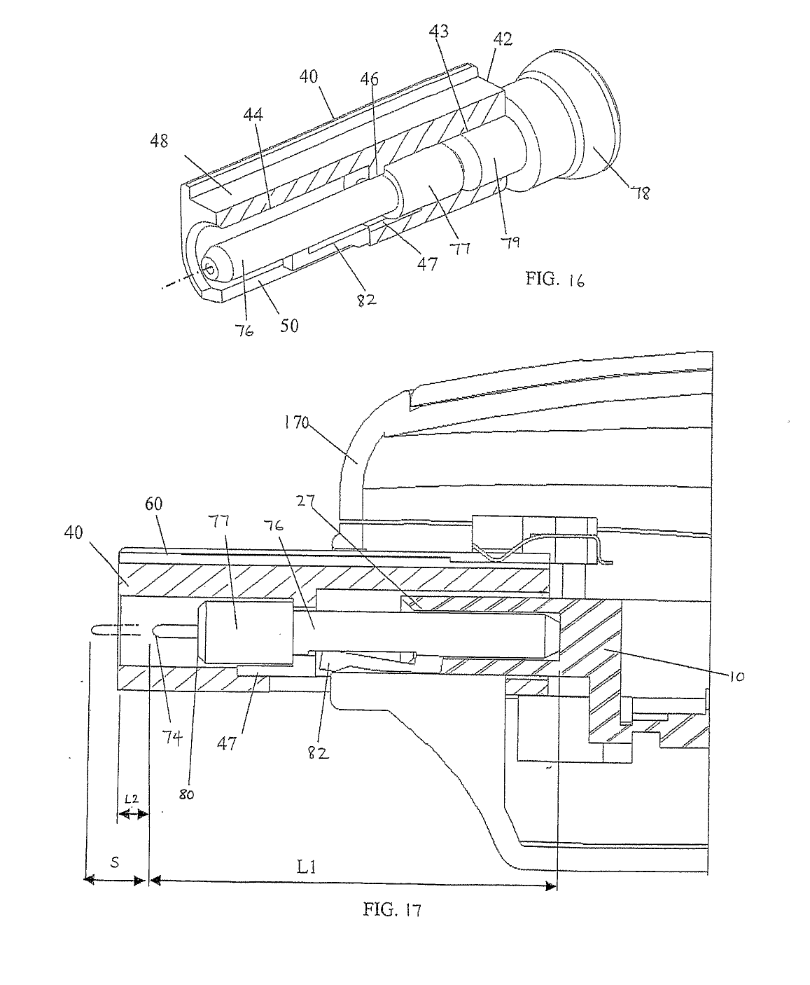

FIG. 16 shows a sectional view of the lancet assembly 100 shown in FIG. 13. As shown in FIG. 14, the casing 40 is elongate and has a longitudinal axis 41 along its length. The casing 40 is hollow and has two cylindrical bores 43,44. A collar 46 separates the two cylindrical bores 43,44. The smaller of the cylindrical bore 43 is at the front end 42 of the casing 40. The cylindrical bore 43 is dimensioned to fit with the locating part 79 of the cap 78 to give an interference fit whilst the fit with the body 77 of the lancet 70 is a clearance fit. The larger of the cylindrical bore 44 is dimensioned to accommodate the collar 27 of the probe 10 and the fit between an external dimension of the collar 27 and the cylindrical bore 44 is also clearance fit. The cylindrical bore 44 has a longitudinal slot 50. The longitudinal slot 50 is dimensioned so that the L-shaped catch 82 on the lancet 70 is slidable in the longitudinal slot 50. In an extension of the longitudinal slot 50 but on the inside of the cylindrical bore 43 is a longitudinal groove 47. The length of the groove 47 is dimensioned so that it is longer than the stroke S of the probe 10. As shown in FIGS. 16 and 17, the groove 47 cuts through the collar 46. Diametrally opposite the longitudinal slot 50 is a flat surface 48 on the top of the casing 40 for mounting the analyte test strip 60 thereon.

In use, the lancet 70 is disposed in the hollow casing 40 such that the free end of the L-shaped catch 82 engages with the end edge of the longitudinal slot 50 so that the lancet 70 becomes locked onto the casing 40 as one assembly. From FIG. 16, it is seen that the free end of the L-shaped catch 82 engaging with the end edge of the longitudinal slot 50 causes the step 84 on the lancet 70 to press against the collar 46 on the casing 40. The fit between the collar 46 and the body 76 of the lancet 70 is also clearance fit and the concentricity of the body 76, 77 of the lancet 70 with the longitudinal axis 41 is maintained by the interference fit between the locating part 79 of the cap 78 that is journalled in the cylindrical bore 43.

FIG. 17 shows a part sectional view of the lancet assembly 100 coupled to the probe 10 of the integrated device 170 according to an embodiment of the present invention. The cap 78 has been sheared off at the notch 80 and the pointed tip 74 of the lancet needle 72 is exposed. The fit between the collar 27 at the free end the probe 10 and the cylindrical body 76 of the lancet 70 is an interference fit. This interference fit allows the lancet 70 to be retained in the probe 10 so that the lancet 70 and probe 10 move as one body during lancing. In addition, this interference fit and the clearance fit around the lancet body 76,77 allow the lancet 70 to take on the characteristic movements of the probe 10 during lancing as described above. In use, the free end of the cylindrical body 76 of the lancet 70 is fully inserted or bottoms-out in the collar 27 when the lancet assembly 100 is fully inserted into the integrated device 170. This bottoming-out of the lancet 70 in the collar 27 allows a penetration depth of the lancet that is predetermined via a depth penetration mechanism provided in the integrated device 170.

By providing an interference fit between the lancet body 76 and the collar 27 of the probe, there is no slipping of the lancet from the collar 27 and therefore the amount of travel of the needle pointed end 74 into a user's skin is substantially determined by the depth penetration mechanism. The interference fit between the lancet 70 and the collar 27 also ensures that substantial concentricity of the lancet 70 with the longitudinal axis 41 is maintained and the pointed end 74 of the lancet takes on the characteristic movement of the probe 10, in terms of displacement, velocity and acceleration as described above. The clearances between the lancet body 76 and the collar 46 and that between the lancet body 77 and the bore 43 also ensure that the pointed end 74 of the lancet takes on the characteristic movement of the probe 10.

In addition, when the lancet assembly 100 is fully inserted into the integrated device 170, sensing terminals T of the test meter provided in the integrated device 170 come into contact with and ride on the terminal end 62 of the lancet assembly 100. A tongue or rib Q at the receptacle R of the integrated device 170 as shown in FIG. 12 disengages or unlocks the L-shaped catch 82 from the end wall of the longitudinal slot 50 of the casing 40. In this unlocked position of the L-shaped catch 82, the L-shaped catch 82 and the entire lancet 70 is uninhibited in its movement but takes on the characteristic movement of the probe 10 when the firing mechanism of the lancing device is activated. After firing of the lancing device, the probe 10 returns to its unprimed position, at which point the lancet 70 and the needle pointed end 74 are withdrawn into the casing 40. At the same time, the L-shaped catch 82 returns to its unlocked or disengaged position.

To discard the used lancet assembly 100, the user pulls on the lancet casing 40 to free the entire lancet 70 from the collar 27 whilst the L-shaped catch 82 is still disengaged. Once the lancet assembly 100 is removed from the receptacle R, the L-shaped catch 82 springs back to its locked position and thereby locks the used lancet 70 inside the casing 40 to prevent egress of the lancet tip 24 from the casing 40 after use. The relocking of the used lancet 70 into the casing 40 minimizes accidental pricking by the lancet tip 24. The L-shaped catch 82 and the longitudinal slot 50 thus form locking adaptations on the casing 40 and on the lancet body 76 for preventing egress of the lancet tip 74 from the casing 40 after use.

In this embodiment, the maximum projection of the needle pointed end 74 from the front face 42 of the casing 40 is given by the stroke S of the probe 10 minus L2.

Depending on the skin characteristics at the intended blood sampling point, for example, thickness and hydration of the epidermis, the depth of wound puncture is a function of S minus L2.

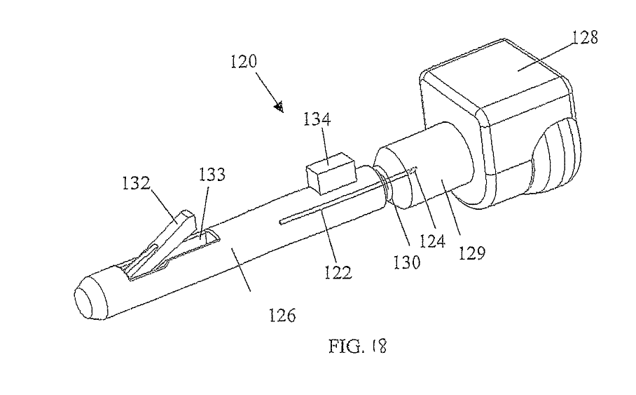

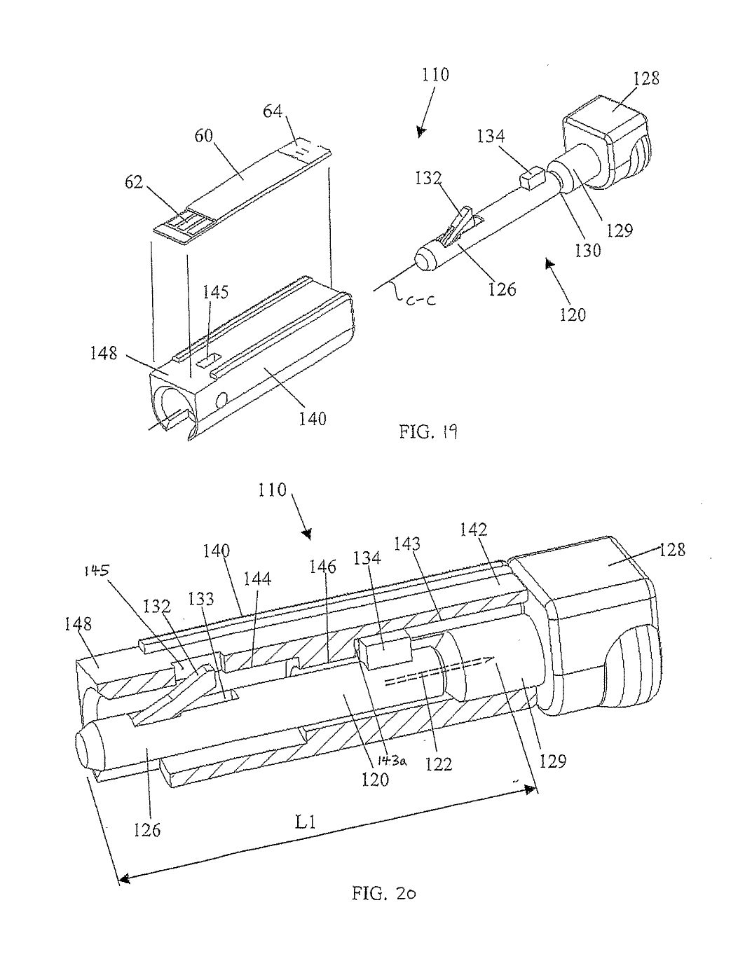

FIG. 19 shows a lancet assembly 110 according to another embodiment of the present invention. The lancet assembly 110 is an assembly of a lancet 120, a casing 140 and an analyte test strip 60. As shown in FIG. 19, the lancet 120 is disposed inside the casing 140 and the test strip 60 is disposed on the casing 40. The lancet 120 includes a needle 122 that is sterilized and insert molded with a thermoplastic just like the earlier lancet 20. As shown in FIG. 19, the needle pointed end or lancet tip 124 is molded within a cap 128. The cap 128 is connected to the molded lancet body 126 by a notch 130 of reduced cross-section. In between the cap 128 and the notch 130 is a locating part 129. The locating part 129 is cylindrical and concentric with the needle 122. On the lancet body 126 but near to the notch 130 is a stopper 134. The stopper 134 projects from the cylindrical surface of the lancet body 126. Also on the lancet body 126 but near to the free end of the lancet body 126 is a catch 132. The catch 132 is extended in its unactivated or locked position and lies on the same meridian as the stopper 134. The catch 132 is operable to deflect into its cavity 133 so that the catch 132 lies within the cylinder surface of the lancet body 126. Just like the earlier lancet 20, the length of the lancet 120 from the free end of the lancet body 126 to the lancet tip 124 is L1.

FIG. 20 shows a sectional view of a lancet assembly 110 shown in FIG. 19. The casing 140 is similar in length to the earlier casing 40. As in the earlier casing, the casing 140 is also hollow and has two cylindrical bores 144, 146. Front cylindrical bore 146 is at the front end 142 of the casing 140 and has a slot 143 to accommodate the stopper 134. The front cylindrical bore 146 is dimensioned to fit with the locating part 129 of the cap 128 to give an interference fit, while the fit between the stopper 134 and the slot 143 is a clearance fit. Rear cylindrical bore 144 is dimensioned to accommodate the collar 27 of the probe 10 of the integrated device 170 and the fit between the external dimension of the collar 27 and the rear cylindrical bore 144 is a clearance fit. The fit between the lancet body 126 and the front cylindrical bore 146 is also a clearance fit. The rear cylindrical bore 144 has an aperture 145 that opens out to a top side 148 of the casing 140. The aperture 145 is dimensioned to accommodate the catch 132 when the lancet 120 is inserted into the casing 140 and the stopper 134 contacts an end-face 143a of the slot 143.

As with the lancet assembly 100, the fit between the collar 27 at the free end of the probe 10 (as shown in FIG. 17) and the lancet body 126 is an interference fit. This interference fit allows the lancet 120 to be retained in the probe 10 so that the lancet 120 and probe 10 move as one body during lancing. In addition, this interference fit and the clearance fit around the lancet body 126 allow the lancet 120 to take on the characteristic movements of the probe 10 during lancing in terms of displacement, velocity and acceleration. In addition, the bottoming-out of the lancet 120 in the collar 27 allows a penetration depth of the lancet that is predetermined via a depth penetration mechanism (not shown in the figures) to be determinate.

When the lancet assembly 110 is inserted into the collar 27 of the probe 10 of the integrated device 170, a lead-in chamfer at the collar 27 pushes the catch 132 down into its cavity 133 and unlocks the catch 132 from the aperture 145. In the earlier embodiment, when the lancet assembly 100 is inserted into the collar 27, the lancet 70 is in contact with the integrated device 170 through the spigot Q and the L-shaped catch 32. In this embodiment, when the lancet assembly 110 is inserted into the collar 27, the lancet 120 does not contact any part of the test meter 7.

After firing of the probe 10, the probe 10 returns to its unprimed position and the lancet 120 is withdrawn into the casing 140. To discard the used lancet assembly 110, the user pulls on the lancet casing 140 to free the entire lancet assembly 110 from the collar 27. As the user pulls on the lancet casing 140, the end-face 143a of the slot 143 in the casing 140 engages the stopper 134 on the lancet body 126, thereby pulling the lancet 120 out of the collar 27 on the probe 10. Once the lancet 120 is removed from the collar 27, the catch 132 springs back to its inactivated or locked position and projects into the aperture 145 of the casing 140 to lock the used lancet 120 inside the casing 140. The aperture 145 and the catch 132 thus form locking adaptations on the casing 140 and on the lancet body 126 for preventing egress of the lancet tip 124 from the casing 140 after use. Locking the used lancet 120 inside the casing 140 thus minimises accidental pricking by the needle 122 and thus allows for safe disposal of the used lancet assembly 110.

The stopper 134 shown in FIGS. 20 and 19 preferably has a rectangular profile. The catch 132 and the stopper 134 need not lie on the same meridian on the cylindrical surface of the lancet body 126. In another embodiment of the lancet assembly 110, the stopper 134 is a circular step like that of step 84 in the earlier embodiment 100 of the lancet assembly. In another embodiment, it is possible that the aperture 145 lies on another face of the casing 140.

A further preferred alternative embodiment of an integrated device 370 according to the present invention is shown in FIG. 21. The integrated device 370 comprises a housing 302 having a receptacle or opening 303 configured for removably attaching a disposable lancet assembly thereto. The integrated device 370 also comprises a test meter for measuring concentration of an analyte in a blood sample. In this preferred embodiment, the lancing mechanism of the integrated device 370 as shown in FIG. 22 similarly comprises a probe 210 that is provided with a driving slot 229 for engaging a driving pin 236 of a probe actuator 215 so that rotation of the probe actuator 215 in a driving direction (preferably clockwise) about an axis of rotation R-R linearly displaces the probe 210 along a central longitudinal axis C-C of a lancet 307 attached to the probe 210, with the axis of rotation R-R being perpendicular to the axis of linear displacement C-C.

However, the probe 210 is further provided with a priming slot 230 for accommodating the driving pin 236 therein during rotation (preferably anti-clockwise) of the probe actuator 215 to the primed position. The priming slot 230 is configured such that during rotation of the probe actuator 215 to the primed position, there is no linear displacement of the probe 210, thereby eliminating the possibility of the lancet tip displacing to accidentally prick the user while the lancing device is being primed for use. This is achieved by shaping the priming slot 230 as a curved slot 230 having a radius of curvature r that is the same as the distance d that the driving pin 236 is displaced from the axis of rotation R-R of the probe actuator 215.

In the preferred alternative embodiment shown in FIG. 22, instead of providing a damper with the probe actuator 215, a helical coil spring 235 is provided at a rear part of the probe 210 for minimizing vibration of the lancet 307 during linear displacement of the probe 210 when engaged with the lancet 307. One end of the helical coil spring 235 is attached to the base plate 204 of the housing (not shown) while the other end of the spring 235 is attached to the rear part of the probe 210. To achieve a desired velocity profile of the lancet 307, a spring 235 having an appropriate size and elastic modulus may be selected.

The integrated device 370 similarly is provided with two pairs of guides 223 and 224 preferably having a v-shaped profile disposed on the front and rear part of the base plate 204 for locating the probe 210 thereon. Each pair of guides 223 and 224 comprises two sloped surfaces facing upwardly and inwardly for slidably engaging sliding surfaces or profile slides 232 and 233 provided on the probe 210.

Each of the sliding surfaces 232, 233 has a radius of curvature rr centred about a curvature defining axis CDA as shown in FIG. 25, wherein the curvature defining axis CDA is coincident with the central longitudinal axis C-C of the lancet 307 when the lancet 307 is engaged by the probe 210. The sliding surfaces 232, 233 therefore comprise sections of a right circular cylinder RCC sharing a same central longitudinal axis C-C as the lancet 307.

As a result of the curvature defining axis CDA of the sliding surfaces 232, 233 being coincident with the central longitudinal axis C-C of the lancet 307, and also the action of the leaf springs 226 biasing the sliding surfaces 232, 233 against the guides 223, 224, the lancet 307 is prevented from translating in any other direction than in a direction parallel to its central longitudinal axis C-C during linear displacement of the probe 210.

FIGS. 23A and 23B show an alternative preferred embodiment of a disposable lancet assembly 300 for use with the integrated device shown in FIG. 21. The lancet assembly 300 comprises a casing 340 having a test strip 360 disposed on a top surface of the casing 340 and a lancet 307 disposed in the casing 340. One end of the casing 340 is adapted to be removably attached to the housing 302 of the integrated device 301. Sensing terminals (not shown) of the test meter provided in the integrated device 370 come into contact with a terminal end 362 of the lancet assembly 300 when the lancet assembly 300 is fully attached to the housing 302. A sensing end 364 of the test strip 360 is provided to receive a blood sample thereon.

The lancet 307 has a lancet body 326 and a lancet tip 324. A cap 378 is integrally molded with the lancet body 326 for encapsulating the lancet tip 324. As shown in FIG. 23B, the lancet 307 is entirely disposed within the casing 340 before use. Even after the cap 378 is broken off in preparation for use, the lancet tip 324 remains within the casing 340, thereby preventing accidental needle stick injury. The lancet tip 324 only emerges from the casing 340 when the lancet assembly 300 has been attached to the housing of the lancet 307 and the lancet 307 engaged by the probe 210 is linearly displaced due to rotation of the probe actuator 315 in the driving direction, upon actuating the integrated lancing device 370.

Locking adaptations 373 are preferably provided on the lancet body 326 and on the casing 340 for preventing egress of the lancet tip 324 from the casing 340 after use. The locking adaptations 373 may comprise cantilever arms as shown in FIG. 23A for engaging appropriately configured recesses provided on corresponding inside surfaces of the casing 340.

To allow for adjusting maximum displacement of the lancet 307 from the lancet assembly 300 during displacement of the probe 210 when the probe is engaged 210 with the lancet 307, the base plate 204 may be configured to be moveable relative to the housing 302 in a direction parallel to the central longitudinal axis C-C of the lancet 307. Maximum depth penetration of the lancet 307 can then be set to a desired level by the user moving the base plate 204 to an appropriate position along the housing 302. This controls the extent of protrusion of the lancet tip 324 from the casing 340 during lancing.

An advantage of using an integrated lancing and testing device 170, 370 (comprising a test meter and the lancing mechanism of the device of FIG. 1 or FIG. 22) together with the lancet assembly 100, 110, 300 is that the number of steps in carrying out an analysis of an analyte in one's blood sample is fewer than those required for a conventional device. For example, in a conventional device, the steps involve in analyzing one's blood glucose level are as follows:
1. removing a new lancet from its container;
2. removing a cover on a lancing device;
3. inserting the new lancet into the lancing device;
4. removing the lancet safety cap;
5. putting back the cover onto the lancing device;
6. priming the lancing device;
7. removing a new test strip from its container;
8. inserting the new test strip on a test meter;
9. lancing a sampling area with the lancing device to make a skin puncture;
10. allowing a droplet of blood to ooze out from the skin puncture;
11. applying the blood sample onto the test strip and obtaining a reading on the test meter;
12. discarding the used test strip from the test meter;
13. removing the cover from the lancing device;
14. putting back the safety cap onto the used lancet;
15. removing the used lancet from the lancing device; and
16. replacing the cover onto the lancing device.

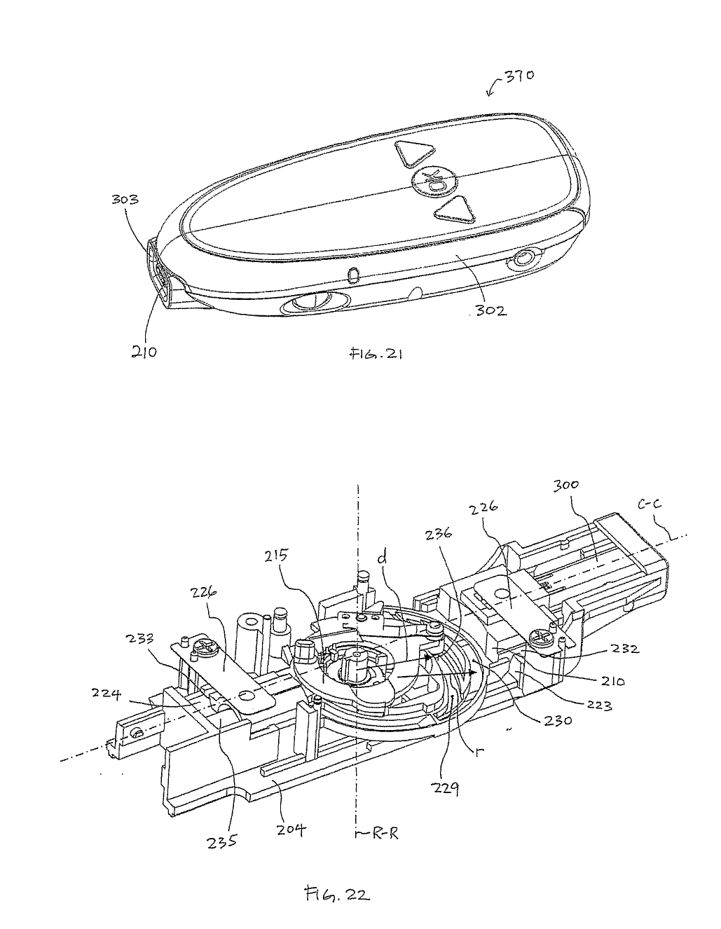

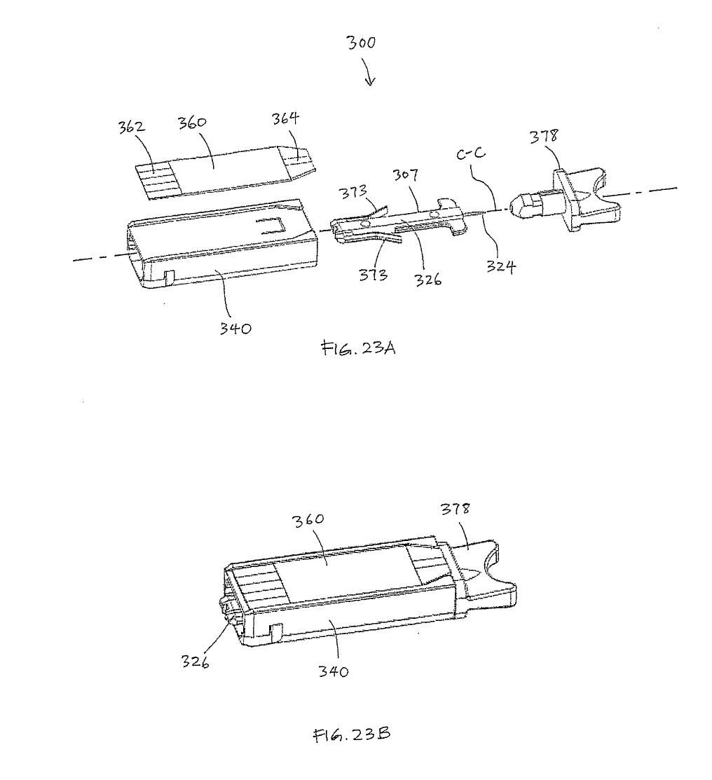

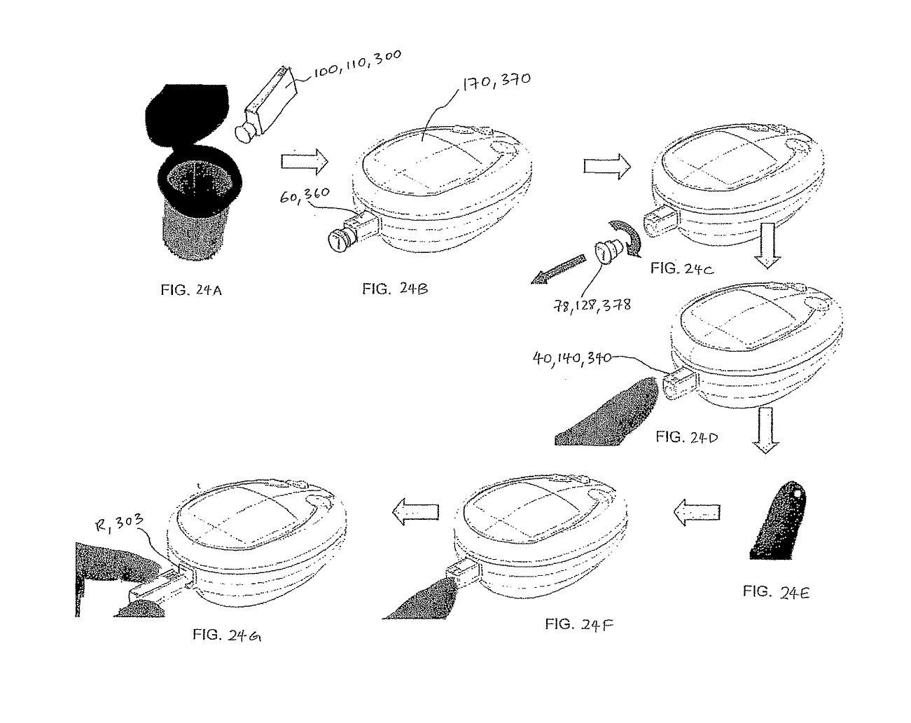

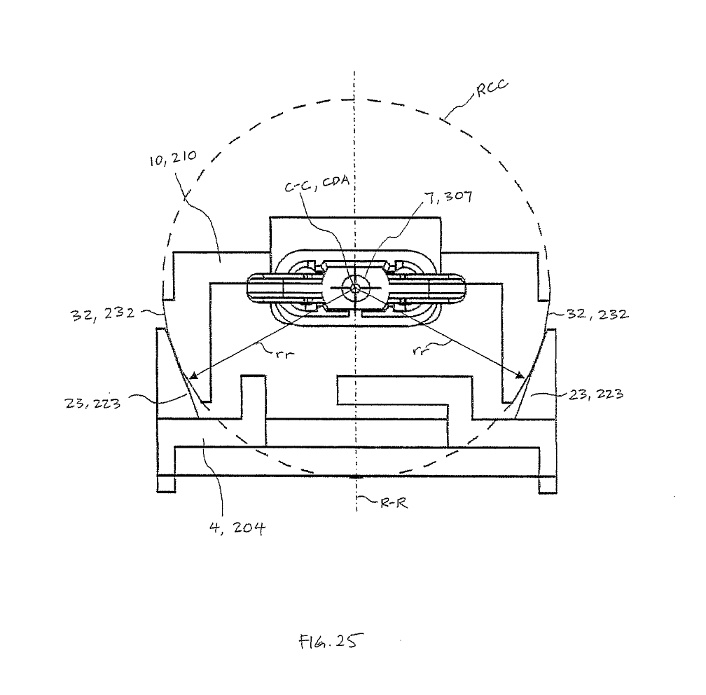

In contrast, in the present invention, the number of steps required to conduct an analysis of one's blood sample, as shown in FIGS. 24A-24G, have accordingly been reduced to seven steps as follows:
1. removing a lancet assembly 100, 110, 300 that has an integral test strip 60, 360 from its container;
2. inserting the lancet assembly 100, 110, 300 into the opening R, 303 of the integrated device 170, 370;
3. tearing away the protective cap 78, 128, 378 from the lancet assembly 100,110, 300;
4. priming and firing the lancet mechanism in the integrated device 170, 370 to puncture one's skin;
5. allowing a droplet of blood to ooze from the skin puncture;
6. transferring the droplet of blood onto the sensing end of the test strip and allowing the test meter in the integrated device 170, 370 to generate a reading; and
7. after the test is completed, removing the lancet assembly 100, 110, 300 from the integrated device 170, 370 for disposal.

Although the number of steps have been reduced, there is no substantive change that a user has to learn in using the analyte test device 170, 370 of the present invention. Another advantage of the present invention includes relocking of a used lancet 20,120, 307 inside the casing 40,140, 340 so that the used lancet assembly 100,110, 300 can be disposed of in a safe manner. When using a conventional lancing device, the skin puncture point is close to the lancet cover and periodic cleaning of blood stains is necessary. With the present invention, the relative distance from the skin puncture point and integrated device 170 is greater than the skin puncture point to the conventional lancing device, so there is little likelihood of blood stain on the test meter; thus, there is no need for periodic cleaning to remove blood stains off the test device. In addition, as the lancing mechanism is provided together with the test meter in the integrated device 170, 370, there is no additional cleaning of a separate lancing device; if there is blood stain, it is likely to appear on the used lancet devices 100,110, 300, which are disposed of. The unused lancet assemblies 100,110, 300 together with the test strips 60, 360 are preferably kept in an air-tight container in compliance with manufacturer's directions so that reliability of the test strips is maintained.

Another advantage of the present invention is that it allows a user control over the individual steps in collecting a blood sample. For example, a user may be used to milking one's finger to ooze out a droplet of blood. The user of the present invention is able to do so after firing the lancing mechanism in the integrated device 170 370; once a sufficient amount of blood has been oozed out, the blood droplet is transferred onto the analyte sensing end 64, 364 of the test strip 60, 360. In the event of a user not being able to obtain a sufficient amount of blood when using some known diagnostic devices, for example a fully automatic diagnostic device, the device has to be primed again to make another skin puncture and often resulting in a test strip being wasted; instead, with the present invention, the user can milk one's finger to obtain a sufficient amount of blood or re-prime the lancing mechanism to make another skin puncture, albeit deeper penetration, without wasting the lancet assembly 100,110, 300 that has been inserted into the integrated device 170, 370.

It should be appreciated that the invention has been described by way of example only and that various modifications in design and/or detail may be made without departing from the scope of this invention. For example, the probe actuator could be configured such that the priming direction and the driving direction are both rotation in the same direction, i.e., both clockwise or both anti-clockwise. Instead of the probe actuator being a rotating actuator biased by a torsion spring and engaging the driving slot on the probe, the probe actuator could comprise an electric linear actuator directly attached to the probe. Instead of two separate sliding surfaces being provided on the probe for engaging a pair of guides on the base plate as shown in the figures, a single continuous curved surface may be provided for engaging the pair of guides such that two areas forming two sliding surfaces on the single curved surface contact the pair of guides. Besides providing a damper or a spring to minimize vibration of the lancet during linear displacement of the probe when engaged with the lancet, other appropriate means such as a resilient foam may be used.

1. A lancing device for use with a lancet for obtaining a blood sample, the lancing device comprising:
a housing;
a probe disposed in the housing; and
a probe actuator for linearly displacing the probe, the probe being configured for releasably engaging a lancet, the probe being provided with sliding surfaces for slidably engaging a pair of guides;
wherein each sliding surface has a radius of curvature centred about a curvature defining axis, the curvature defining axis being coincident with a central longitudinal axis of the lancet when the lancet is engaged by the probe; and
wherein the sliding surfaces are continually biased against the pair of guides such that when the lancet is engaged by the probe, the lancet is prevented from translating in any other direction than in a direction parallel to its central longitudinal axis during linear displacement of the probe.

2. The lancing device of claim 1, wherein the pair of guides comprises two sloped surfaces, each of the sloped surfaces being disposed on either side of the curvature defining axis.

3. The lancing device of claim 2, wherein the probe, the probe actuator and the pair of guides are disposed on a base plate of the housing, the base plate being moveable relative to the housing in a direction parallel to the central longitudinal axis of the lancet for adjusting maximum displacement of the lancet from the lancet assembly during displacement of the probe when the probe is engaged with the lancet.

4. The lancing device of claim 3, further comprising a second pair of guides and further sliding surfaces provided on the probe for slidably engaging the second pair of guides.

5. The lancing device of claim 4, wherein the probe actuator is rotatable about a pivot disposed on the base plate of the housing with an axis of rotation perpendicular to the central longitudinal axis of the lancet.

6. The lancing device of claim 5, wherein the probe actuator comprises a driving pin disposed at a distance from the axis of rotation for engaging a driving slot provided in the probe, the driving slot being configured such that rotation of the probe actuator in a driving direction results in linear displacement of the probe.

7. The lancing device of claim 6, further comprising a priming slot provided in the probe for accommodating the driving pin therein during rotation of the probe actuator to a primed position, the priming slot being configured such that rotation of the probe actuator to the primed position results in no linear displacement of the probe.

8. The lancing device of claim 7, further comprising a priming actuator for rotating the probe actuator to the primed position.

9. The lancing device of claim 8, further comprising a biasing element for biasing the probe actuator towards rotating in the driving direction, and further comprising an actuating button configured for releasing the probe actuator from the primed position such that when the actuating button is pressed, the probe actuator rotates in the driving direction under the bias of the biasing element.

10. The lancing device of claim 9, further comprising means for minimizing vibration of the lancet during linear displacement of the probe when engaged with the lancet.

11. The lancing device of claim 10, wherein the housing is configured for removably attaching a disposable lancet assembly thereto, the disposable lancet assembly comprising the lancet, and wherein the probe is configured for releasably engaging the lancet when the lancet assembly is attached to the housing.

12. The lancing device of claim 11, further comprising an analyte test meter for determining concentration of an analyte in the blood sample.

13. A disposable lancet assembly for use with a lancing device, the lancet assembly comprising:
a casing configured for releasable attachment to a housing of the lancing device;
a lancet contained in the casing, the lancet being configured for releasable engagement with a linearly displaceable probe of the lancing device, the lancet being moveable with respect to the casing when the lancet is engaged with the linearly displaceable probe; and
a test strip disposed on the casing for receiving a blood sample thereon.

14. The disposable lancet assembly of claim 13, further comprising locking adaptations on the casing and on the lancet body for preventing egress of a tip of the lancet from the casing after use.

15. The disposable lancet assembly of claim 13, wherein he test strip comprises a sensing end for receiving the blood sample and a terminal end for contacting sensing terminals of a test meter provided with the lancing device.

16. A lancing kit comprising the lancing device of claim 12 and the disposable lancet assembly comprising:
a casing configured for releasable attachment to a housing of the lancing device;
a lancet contained in the casing, the lancet being configured for releasable engagement with a linearly displaceable probe of the lancing device, the lancet being moveable with respect to the casing when the lancet is engaged with the linearly displaceable probe; and
a test strip disposed on the casing for receiving a blood sample thereon.

17. A method for determining an analyte in a blood sample, said method comprising:
inserting a lancet assembly into an integrated device comprising a lancing device and a test meter, the lancet assembly comprising a lancet disposed within a casing and a test strip disposed on the casing, said lancet and casing comprising locking adaptations for preventing egress of a tip of the lancet from the casing after use;
actuating the lancing device to lance a finger with the lancet;
collecting a blood sample from the lanced finger;
transferring the blood sample onto the test strip and obtaining a reading from the test meter; and
removing the lancet assembly from the integrated device and simultaneously locking the lancet within the casing.
